(19) United States
(12) Patent Application Publication
(10) Pub. No.: US 2005/0165344 A1
(43) Pub. Date: Jul. 28, 2005

(54) Method and apparatus for treating heart failure

(76) Inventor: John D. Dobak III, San Diego, CA (US)

Correspondence Address: GERALD W. SPINKS, P. O. BOX 5242, GLACIER, WA, 98244, US

(21) Appl. No.: 10/996,809

(22) Filed: Nov. 23, 2004

Related U.S. Application Data
(60) Provisional application No. 60/615,880, filed on Oct. 5, 2004.
(60) Provisional application No. 60/539,673, filed on Jan. 27, 2004.
(60) Provisional application No. 60/532,983, filed on Dec. 29, 2003.
(60) Provisional application No. 60/525,567, filed on Nov. 26, 2003.

(51) Int. Cl. A61M 5/00

(52) U.S. Cl. 604/8

(57) ABSTRACT

An apparatus for treating heart failure, including a conduit positioned in a hole in the atrial septum of the heart, to allow flow from the left atrium into the right atrium. The conduit is fitted with one or more emboli barriers or one-way valve members, to prevent thrombi or emboli from crossing into the left side circulation.

### CROSS REFERENCE TO RELATED APPLICATIONS

This application relies upon U.S. Provisional Patent Application No. 60/525,567, filed on Nov. 26, 2003, and entitled “Left Atrial Pressure Relief System for CHF”; U.S. Provisional Patent Application No. 60/532,983, filed on Dec. 29, 2003, and entitled “Method for Treating Heart Failure”; U.S. Provisional Patent Application No. 60/539,673, filed on Jan. 27, 2004, and entitled “Method for Treating Heart Failure”; and U.S. Provisional Patent Application No. 60/615,880, filed on Oct. 5, 2004, and entitled “Method for Treating Heart Failure”.

### STATEMENT REGARDING FEDERALLY SPONSORED RESEARCH OR DEVELOPMENT

Not Applicable

### BACKGROUND OF THE INVENTION

1. Field of the Invention

This invention is in the field of prevention or remediation of heart disease.

2. Background Art

The human heart delivers oxygenated blood to the organs of the body to sustain metabolism. The human heart has four chambers, two atria and two ventricles. The atria assist with filling of the ventricles, which pump blood to the body and through the lungs. The right ventricle pumps blood through the lungs to be oxygenated and the left ventricle pumps the oxygenated blood to the body.

A schematic of the heart and the pressures in each chamber is shown in FIG. 1. Pressures are given in mm Hg. The right atrium is indicated at RA, the left atrium is indicated at LA, the right ventricle is indicated at RV, and the left ventricle is indicated at LV. The pulmonary artery is indicated at PA, and the pulmonary capillary wedge pressure is indicated as PCW.

The cardiac pumping cycle is divided into two phases: diastole and systole. Diastole is the period of passive atrial and ventricular filling with blood. Diastole is followed by systole in which the atria, then the ventricles, contract. The atrial contraction pumps an additional volume of blood into the ventricles just prior to ventricular contraction.

A graph of the cardiac filling and pumping cycle, as reflected by the left-sided heart chambers, is shown in FIG. 2. Left atrial pressure is indicated by the line labeled LA, and left ventricular pressure is indicated by the line labeled LV. The electrocardiographic tracing is shown as the curve labeled EKG. During diastole, the mitral valve MV is open, so that the left atrial and left ventricular pressures are equal. In late diastole, left atrial contraction causes a small rise in pressure, a wave labeled “a”, in both the left atrium and the left ventricle. The onset of ventricular mechanical systole is marked by the initiation of left ventricular contraction. As the left ventricular pressure rises and exceeds the pressure of the left atrium, the mitral valve closes, contributing to the first heart sound, labeled “S₁”. As left ventricular pressure rises above the aortic pressure, the aortic valve AV opens, which is a silent event. As the ventricle begins to relax, and its pressure falls below the pressure of the aorta, the aortic valve closes, contributing to the second heart sound, labeled “S₂”. As left ventricular pressure falls further, below the pressure of the left atrium, the mitral valve opens, which is silent in the normal heart. In addition to the “a” wave, the left atrial pressure curve displays two additional positive deflections. The “c” wave represents a small rise in left atrial pressure as the mitral valve closes, and the “v” wave is caused by passive filling of the left atrium from the pulmonary veins during systole, when the mitral valve is closed. The right atrium displays “a”, “c” and “v” waves similar to those shown in FIG. 2.

Heart failure is a medical syndrome characterized by deterioration of cardiac pump function. The primary deterioration is a progressive loss of heart muscle compliance and contractility. Loss of pump function leads to cardiac dilation, blood volume overload, pulmonary congestion, and ultimately organ failure. Symptoms of heart failure include orthopnea, dyspnea on exertion, cough, fatigue, and fluid retention.

There are two types of heart failure. Systolic failure is primarily loss of left ventricular contractility leading to reduced delivery of blood to the body. Systolic failure is associated with a reduced ejection fraction. Normal ejection fraction is greater than 50%. Diastolic failure is due to a loss of compliance of the left ventricle, which limits blood filling during diastole. Typically, there is no reduction in cardiac ejection fraction associated with diastolic failure. As the heart failure syndrome progresses, both systolic and diastolic failure are present.

The mechanisms that cause the heart to fail are thought to be mechanical and neurohumoral. Most commonly there is an insult to the myocardium in the form of a heart attack that causes heart muscle necrosis. This leads to mechanical changes in the heart such as reduced compliance, reduced contractility, or both. The body responds to these changes by activating various neurohumoral pathways, such as the adrenergic system, which leads to remodeling changes that further exacerbate the mechanical derangements. This cycle continues until the heart eventually completely fails.

The primary mechanical change is hypertrophy of the left ventricle or an increase in the thickness of the ventricular muscle. This hypertrophy can be eccentric or concentric, but both are present as the disease progresses. In addition to hypertrophy, the shape of the ventricular chamber changes from that of a prolate ellipse to a more globular shape. The hypertrophy and shape change are thought to be due to an adaptive response related to increases in left ventricular end-diastolic volume (LVEDV) and consequently pressure (LVEDP). Increases in LVEDP ultimately cause increases in left ventricular wall stress. The hypertrophic response and the globular shape help to reduce wall stress.

However, even after the adaptive response, the diseased heart is typically subjected to repeated episodes of increased LVEDP and wall stress. These are typically associated with sudden increases in venous return to the heart, such as may be caused by lying down, exercise, or fluid retention, or that occur during periods of transient ischemia which temporarily reduce compliance.

Because of the direct communication between the left ventricle and left atrium, increases in LVEDP are also associated with commensurate increases in pressure in the left atrium. The left atrium can undergo similar hypertrophy and dilation that ultimately lead to atrial fibrillation, a serious arrhythmia of the heart. In addition, the increases in left atrial pressures lead to an increase in back pressure to the pulmonary circulation. This increased pressure leads to pulmonary edema, or congestion, that causes cough and shortness of breath that can be particularly prominent when lying down or on exertion. Left atrial pressures (LAP) greater than 16 mm Hg are associated with a higher mortality.

One primary objective of heart failure therapy is to reduce LVEDP. The only currently available therapies to accomplish this are drugs such as calcium channel blockers that reduce ventricular compliance (diastolic failure) and diuretics that reduce blood volume. Beta blockers are used to blunt the neurohumoral response to slow the remodeling changes. None of these therapies is effective at preventing disease progression or eliminating pulmonary congestion.

New therapeutic strategies are now being developed to reduce the pressures within the left ventricle (unloading) and/or the stresses on the heart muscle. Ventricular assist devices actively pump blood out of the left ventricle thereby reducing the left ventricular pressure. They have been shown to improve heart function and cause positive remodeling of the left ventricle. Further, by reducing the volume of blood in the left ventricle and consequently the pressure in the left ventricle they greatly improve the symptoms of the heart failure. Passive restraint devices limit dilation of the left ventricle to improve heart function. There is ample clinical data to suggest that the strategy of left ventricular unloading will slow or halt the progression of the disease; however, current approaches and devices require a major surgical procedure to be deployed and/or are complex, costly devices, and are thus reserved for end stage patients.

It is an object of this invention to reduce left atrial pressures and LVEDP and improve the symptoms of heart failure related to pulmonary edema or congestion.

It is a further object of this invention to reduce left atrial pressures and LVEDP and prevent or slow the progression of heart failure.

It is a still further object of this invention to reduce left atrial pressures and LVEDP to prevent and or slow the development of atrial fibrillation.

It is another objective of this invention to create an interatrial septal conduit for the treatment of heart failure and reduce the risk of cryptogenic stroke.

### BRIEF SUMMARY OF THE INVENTION

The invention is a left atrial pressure relief system for reducing left atrial pressures and left ventricular end diastolic pressures (LVEDP). The system consists of an interatrial septal conduit with an emboli barrier or trap mechanism to prevent cryptogenic stroke due to thrombi or emboli crossing the conduit into the left sided circulation. A wire mesh may serve as one emboli barrier design. Alternatively, a one-way valve with an opening pressure of at least 1 mm Hg may be used to reduce stroke occurrence. The direction of flow through the valve is from the left atrium to the right atrium. The conduit allows the shunting of blood from the left atrium to the right atrium. The diameter of the conduit allows flow rates of 250 to 1,500 ml/min across the atrial septum depending on the left to right atrial pressure gradient. The shunting of blood will reduce left atrial pressures, thereby preventing pulmonary edema and progressive left ventricular dysfunction. The conduit will also reduce LVEDP.

The novel features of this invention, as well as the invention itself, will be best understood from the attached drawings, taken along with the following description, in which similar reference characters refer to similar parts, and in which:

### BRIEF DESCRIPTION OF THE SEVERAL VIEWS OF THE DRAWINGS

FIG. 1 is a section view of a heart, and a schematic of the flow path of the blood;

FIG. 2 is a graph of the cardiac filling and pumping cycle;

FIG. 3 is a section view of a first embodiment of the apparatus of the present invention;

FIG. 4 is a perspective view of a second embodiment of the apparatus of the present invention;

FIGS. 5a and 5b are partial section views of a third embodiment of the apparatus of the present invention;

FIG. 6 is a section view of a fourth embodiment of the apparatus of the present invention;

FIGS. 7a and 7b are plan and side elevation views of a fifth embodiment of the apparatus of the present invention;

FIG. 8 is a side elevation view of a sixth embodiment of the apparatus of the present invention;

FIGS. 9a and 9b are side elevation views of a seventh embodiment of the apparatus of the present invention; and

FIG. 10 is a partial section view of an eighth embodiment of the apparatus of the present invention.

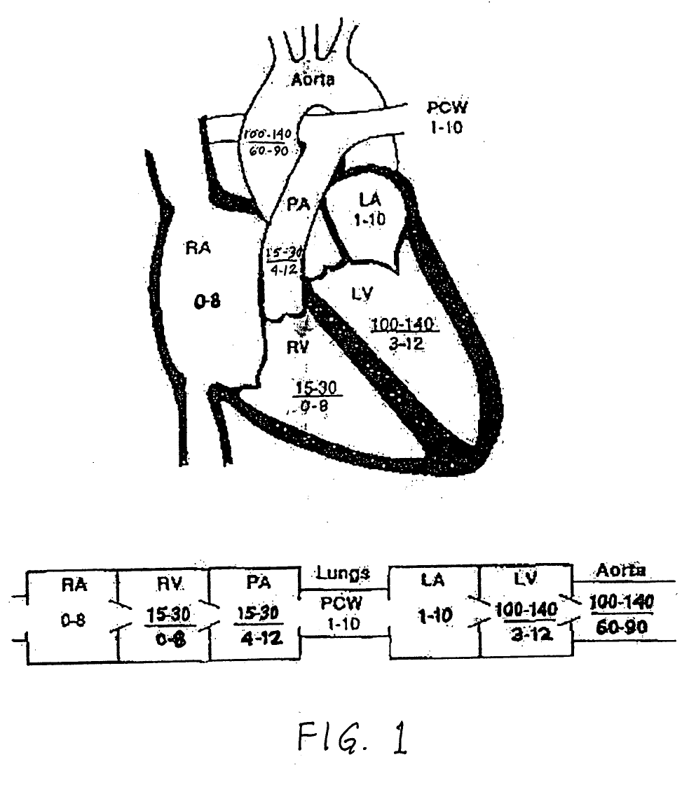

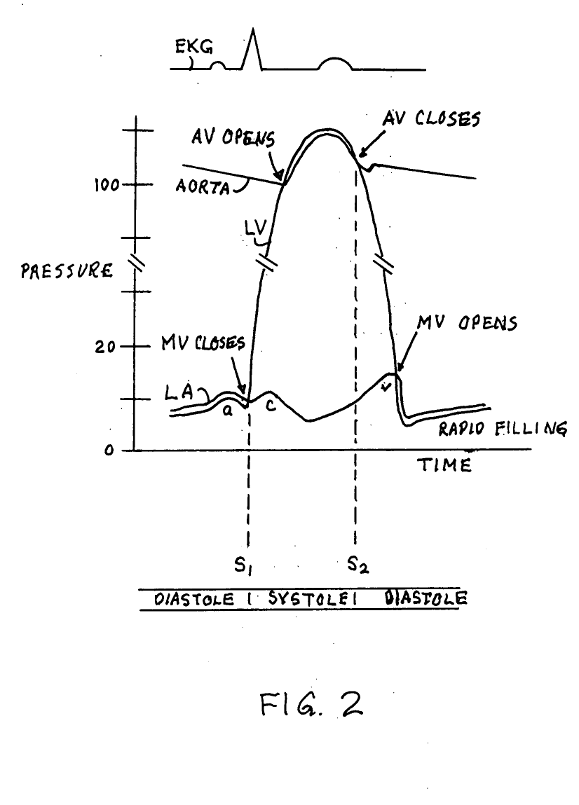

### DETAILED DESCRIPTION OF THE INVENTION

Heart failure is characterized by increased left heart pressures (ventricular and atrial), which cause symptoms of pulmonary congestion and deterioration of left ventricular function. These left sided pressures exceed right sided pressures. Consequently, a conduit positioned in the atrial septum would allow blood flow to shunt from the left atrium to the right atrium, thereby reducing left atrial and left ventricular pressures. The general therapeutic concept occurs naturally in a condition known as Lutembacher's syndrome. Lutembacher's syndrome is the simultaneous occurrence of mitral valve stenosis and an atrial septal defect. Typically, mitral valve stenosis causes severe left atrial pressure increases; however, in Lutembacher's syndrome these pressure increases are prevented by the atrial septal defect, and patients may remain relatively asymptomatic for pulmonary congestion.

Atrial septal defects are a congenital anomaly. Small defects are often asymptomatic and may not require treatment. Large defects may lead to symptoms of right heart failure, but only after many decades. Consequently, large defects are often closed by surgery, or with catheter based closure devices, when detected. However, large and small septal defects are associated with the risk of cryptogenic stroke or ischemia. This occurs when a thrombus or embolus from the right sided circulation crosses the defect and enters the left sided circulation. This thrombus or embolus can then occlude an arterial vessel causing end organ (heart, brain, kidney, etc.) ischemia and damage.

The concept of left atrial pressure reduction by interatrial shunting was rigorously studied in healthy dogs (Roven, et. al., The American Journal of Cardiology, 24: 209, 1969). In this study, interatrial communications were shown to reduce left atrial pressures by 30% to 50%. Importantly, this study showed that increasing interatrial flow from the left to the right does not result in an increase in right atrial pressures which would tend to reduce flow and cause right sided symptoms of congestion. Rather, right atrial pressures remain normal while blood flow through the lungs increases to accommodate the increased interatrial shunt flow. This produces a sustained reduction of left atrial pressures at various shunt flows.

Today, interatrial communications by atrial septostomy are created in congenital heart defects such as hypoplastic left ventricle, where life threatening left atrial pressure increases occur (Cheatham, Journal of Interventional Cardiology, 14 (3): 357, 2001). In some cases, a coronary stent is placed across the septum to prevent closure. The devices used in this procedure do not address the concern for cryptogenic stroke or ischemia. In addition, some patients with severe congestive heart failure are placed on extracorporeal membrane oxygenation and given an interatrial communication to reduce left atrial pressure and its attendant pulmonary congestion, again without addressing the issue of cryptogenic ischemia.

In U.S. Patent Application Publication U.S. 2002/0173742 A1, by Keren, et al., a catheter deployed interatrial conduit is disclosed for treating heart failure and severe pulmonary congestion. This application describes a conduit with a valve incorporated centrally and with various methods (struts and spiral ribbons) for retaining the conduit to the septum. While a valved design may reduce the risk of cryptogenic ischemia, such a design may not be optimal due to a risk of blood stasis and thrombus formation on the valve. In addition, valves can damage blood components due to turbulent flow effects. Other embodiments disclosed in this patent application publication do not contain a valve; however, these non-valved designs do not have a method or mechanism for reducing cryptogenic ischemia, such as an emboli barrier or trap. Additionally, there is no method or mechanism disclosed to allow the gradual increase or opening of flow across the conduit.

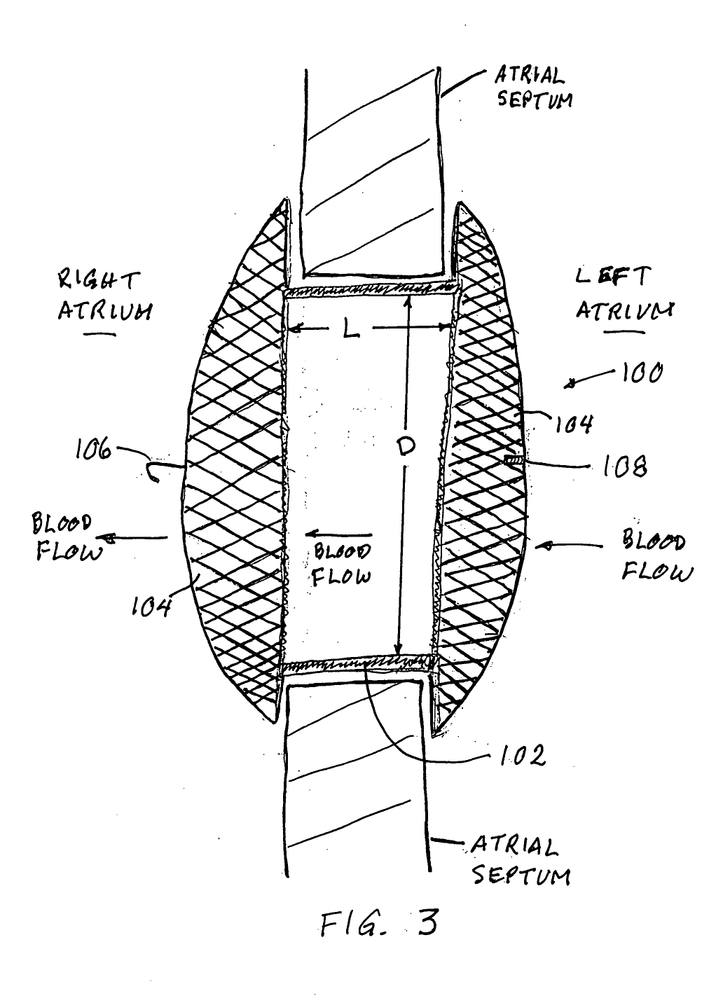

Thus, as shown in FIG. 3, the preferred embodiment 100 of the present invention includes a conduit 102 deployed between the left atrium and the right atrium that allows the desired left to right shunting, but reduces the risk of cryptogenic ischemia, without the need for a valve. This can be accomplished by a tubular conduit 102, with an embolic filter 104 on either end or both. The pore size of the embolic filters 104 can be in the range of 0.1 to 2.0 mm. A deployment hook 106 can be provided on the right atrial side of the device 100, or a threaded stylet connector 108 can be provided on the left atrial side of the device 100.

The conduit 102 of this design is a tubular structure (2 to 10 mm diameter, preferred, or larger) that spans the atrial septum. The conduit flow diameter D would be wide enough to allow sufficient blood flow across it to reduce the left atrial pressure. The deployed diameter D of the conduit 102 would be optimized to reduce jet/turbulent flow effects and shear forces which may damage blood cells and components, and atrial tissue, based on the anticipated flow through the conduit 102. Conduit sizes of 6.0 to 10.0 mm can reduce these turbulent effects.

Preferably, the cross sectional area of the conduit 102 would not exceed 2.0 cm² and would remain typically at less than 1.0 cm². A larger conduit (greater than 2.0 cm²) would likely result in bidirectional flow which may limit the left atrial pressure reduction effect. Also, a larger conduit can result in an excessive volume of blood shunting, which can cause left ventricular diastolic dysfunction due to right ventricular volume overload and interventricular septal shift.

Preferably, the conduit 102 could be opened slowly (over 6 hours, to several days or weeks), after initial placement, as sudden shunting of blood may result in a drop in stroke volume and consequently a reduction in cardiac output. This may be particularly important in patients with substantial systolic dysfunction. These patients may rely more on high LVEDP pressures to maintain the left ventricle's stroke volume.

The flow rate through the conduit 102 at any given time would be determined by the left to right atrial differential pressure and the conduit diameter. The left to right atrial pressure gradient is dynamic and constantly changing based on conditions such as ventricular compliance, patient blood volume status, and venous return. A conduit diameter of 3 to 10 mm would allow flow rates of 500 ml/min to 2000 ml/min at pressure gradients of 5 mm Hg to 12 mm Hg across the atrial septum. Consequently, a conduit could be self-regulating to meet changing demands over time.

The deployed length L of the conduit 102 would be approximately equivalent to the thickness of the atrial septum, which may be as thin as 1.0 mm to as thick as several millimeters. Ideally, the conduit portion of the device 100 is designed to self adjust to the thickness of the septum by shortening or lengthening. One way to accomplish this is to use a coiled or spring type design for the conduit 102. During deployment, the coiled conduit 102 would be stretched long and to a smaller diameter D. Upon deployment, the length L of the coil conduit 102 would shorten, and the diameter D would enlarge, and thereby adjust the length L to the atrial septal thickness. Alternatively, the septal thickness could be determined using an imaging modality such as ultrasound and an appropriate conduit length L would be chosen.

Depending on the desired diameter D of the conduit 102, the tubular structure could be a rigid tube or an expandable tube. Tube diameters of 2.0 mm to about 5.0 mm could use a non-expandable structure, whereas diameters greater than about 7.0 mm would require an expandable structure. An expandable structure could be similar to a coronary stent design and could be balloon expandable or self-expandable. Both balloon expandable and self-expandable tubular structures are well known to those skilled in the art of implantable medical products.

Preferably, a self-expandable embodiment 200 would be used, which would expand due to the presence of a filter 204 on the end of the tube 202, as shown in FIG. 4. A polyester, Goretex™, or Dacron™ graft/sheath 210 could be placed around and sewn onto the tubular structure, such as an expandable wire frame 212, or within the tubular structure 212, to prevent blood leakage and promote endothelialization.

To prevent cryptogenic stroke, filters or traps or wire mesh structures 204 can be placed on both ends or on one end of the tubular structure 212. The wire filter/mesh or emboli barriers would prevent large emboli from crossing the septum and entering the left sided circulation. The barriers 204 could be integral to the tubular structure and could serve to anchor the tube 202 across the septum. If a barrier 204 were used on only one end, such as the right end, a strut 214 for anchoring the conduit 202 to the atria on the left end would be used. This strut 214 could be designed as a spiral wire or ribbon, laser cut from one end of the tubular conduit 202, as shown in FIG. 4. The spiral ribbon 214 could subsequently be shaped to expand and flatten against the septum to a size larger than the tubular conduit 202, thereby anchoring the conduit 202. There are several strut designs that could be employed for anchoring a device to the atrial septum, and the spiral ribbon design described above is illustrative.

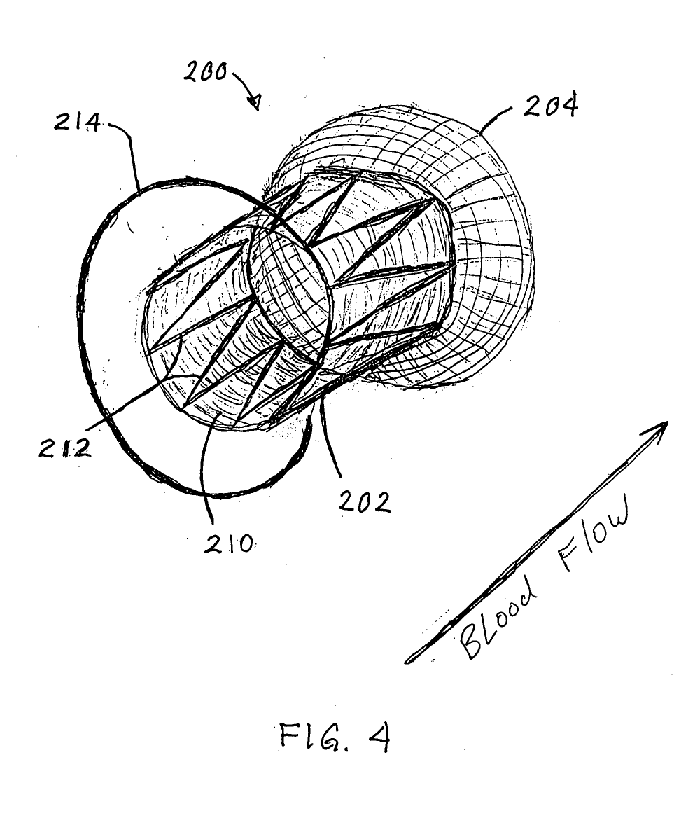

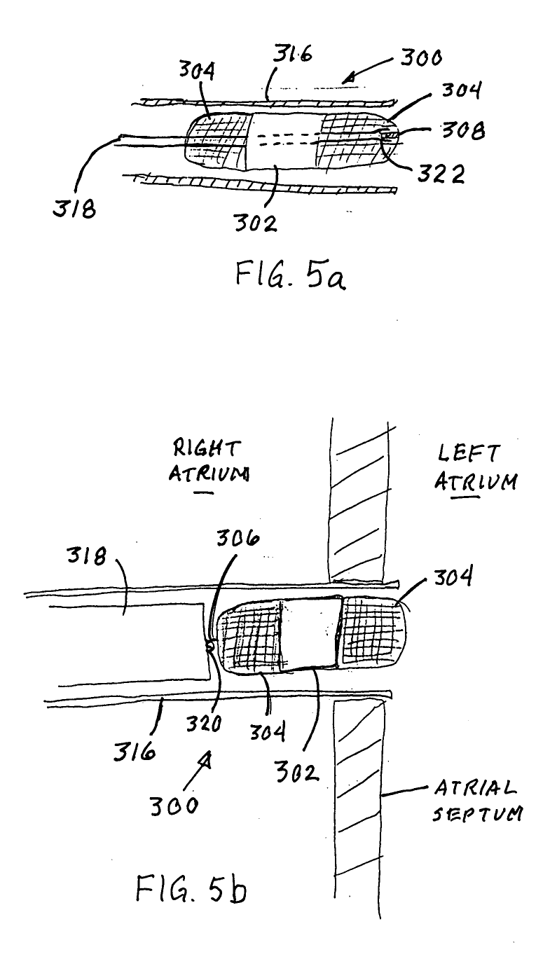

As in the embodiment 300 shown in FIGS. 5a and 5b, the filter 304 can be a mesh-like design that would collapse into a transseptal delivery catheter 316 and would deploy by expanding larger than the tube conduit 302. The embolic barrier would have a pore size of 0.1 to 2.0 mm or greater. A wire mesh design could flatten out against the septum or remain globular on each end. Alternatively, a porous polymer supported on expandable struts could also serve as a barrier. Alternatively, a flat spiral design could be deployed that would also anchor the conduit to the septum. The spiral would have 0.1 to 2.0 mm spacing between successive turns. Other mechanisms to filter or prevent thrombi from crossing the conduit from the right to the left could be employed.

A mechanism for attaching the device 300 to a stylet 318, that would be used to push and pull the device 300 during deployment, would be connected to the right or left atrial filter/mesh structure 304 or both. One embodiment is a threaded extension 308, 1.0 mm to several millimeters long, as shown in FIG. 5a. The distal end of the stylet 318 could then be attached to the device 300 by screwing the threaded extension 308 into a threaded receptacle 322 in the distal end of the stylet 318. Alternatively, as shown in FIG. 5b, a hook mechanism 306 could be utilized. The hook 306 could be captured with a wire loop 320 on the stylet 318.

In one embodiment, the attachment mechanism, such as the threaded connector 308, is located on the left atrial filter mechanism 304 and protrudes inward toward the conduit 302. This results in pulling the filter mesh 304 internally to the conduit 302 during deployment. Subsequently, the mesh 304 is pushed out with the stylet 318 into the left atrium during deployment.

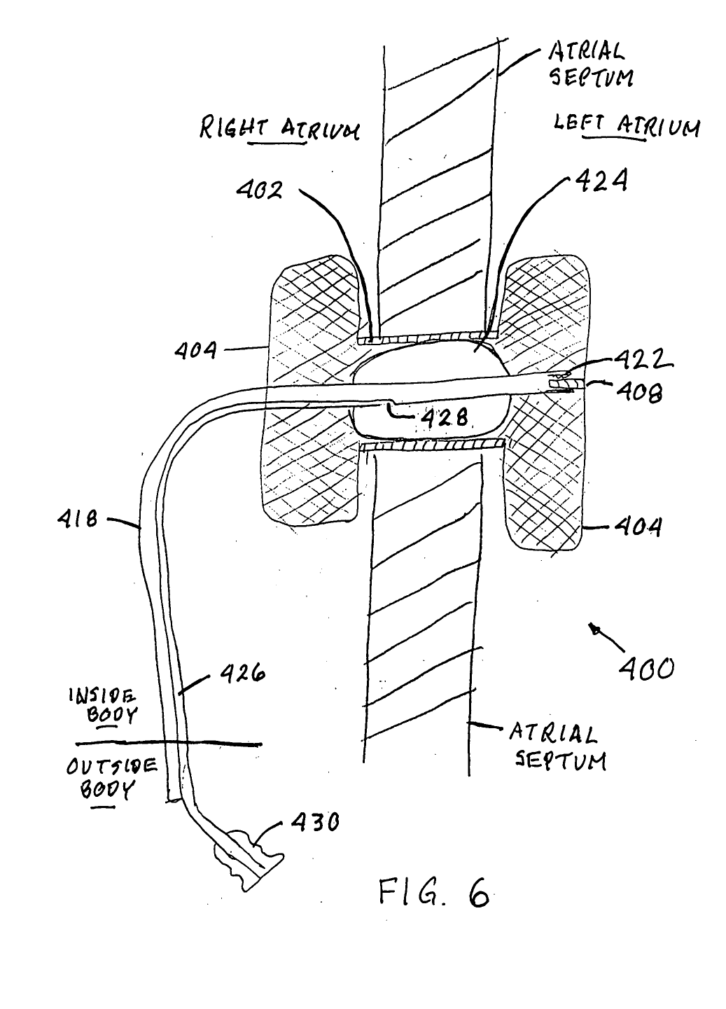

As seen in the embodiment 400 of FIG. 6, to control the opening of the conduit 402 after placement of the device 400, the stylet 418 in some embodiments may have an expandable and collapsible balloon 424 on the section of the stylet 418 that resides substantially within the conduit 402 and between the filters 404. As before, the connector 422 on the distal end of the stylet 418 would be threaded onto the threaded extension 408 on the left end filter 404. The interior of the balloon 424 would be in fluid communication with a balloon inflation channel 426 within the stylet 418. This channel 426 would be in fluid communication with a balloon inflation port 428 that would allow saline to be delivered to or withdrawn from the balloon 424, using a syringe 430 or some other fluid injection and withdrawal device. This would allow the balloon 424 to be inflated and deflated as necessary. This balloon 424 may also be used to expand the conduit 402 in the balloon expandable designs. Preferably, the balloon 424 is elastic, so that in its collapsed form it lies flat against the stylet 418, thereby minimizing the profile/diameter of the stylet 418 and facilitating removal from the device 400 and from the body.

A biocompatible material from which the emboli barrier and conduit could be made is nitinol (nickel titanium alloy) or stainless steel, or other materials used as implantable in the vasculature. These materials are commonly used in implantable medical products and are familiar to those skilled in the art. This material choice may enhance deliverability of the emboli barrier and conduit. The emboli barrier and conduit may be coated with a material, polymer, or chemical to improve blood and tissue compatibility. Heparin is one such chemical. Processes for coating devices to improve blood and tissue compatibility are known to those skilled in the art.

The emboli barrier and conduit would be placed using a transvascular catheter approach. A guide catheter would be placed against the septum on the right atrial side, through either the femoral vein or subclavian or jugular vein. A transseptal needle catheter would be used to puncture through the septum, after which a guide wire would be placed across the septum into the left atrium. Dilation catheters could be slid over the guide wire until the septal hole is large enough to accommodate the delivery catheter (3 to 6 mm diameter). Alternatively, a dilation balloon could be used to expand the size of the initial septal hole. A dilation balloon with cutting blades mounted on the balloon may facilitate enlargement of the septal hole. A cutting dilation balloon is known to those skilled in the art.

After appropriate dilation of the initial septal puncture, the delivery catheter 316 would then be placed across the septum. The interatrial conduit 102, 202, 302, 402 and emboli barrier 104, 204, 304, 404 would be collapsed inside the delivery catheter 316, attached to the delivery stylet 318, 418. The interatrial conduit would then be pushed through the delivery catheter until the left atrial anchoring filter or struts were deployed (expanded). The conduit and the delivery catheter could be pulled back slightly to engage the struts/barrier with the left atrial side of the septum. The delivery catheter alone would then be pulled back to deploy the right atrial septal barrier or mesh. The stylet would then be detached from the device 100, 200, 300, 400.

In situations where it may be undesirable to allow the complete flow of shunting to occur immediately, a balloon 424 on the stylet 418 would be inflated during or at the end of the placement procedure prior to detachment of the stylet 418. When fully inflated, the balloon 424 would prevent the shunting of blood. Preferably, the balloon 424 is inflated using saline or some other biocompatible fluid. Subsequently, over a period of several hours to several days or weeks the balloon 424 would be gradually deflated. This gradual deflation may occur at hourly, daily, or weekly intervals or longer. A syringe 430 or device that can precisely remove a desired volume from the balloon 424 could be used. Such a device may have a pressure sensing and feedback mechanism. The balloon 424 could be deflated while monitoring the cardiac output. Non-invasive devices for monitoring cardiac output are known to those skilled in the art. Once complete deflation of the balloon 424 had occurred, the stylet 418 would be disconnected from the device 400 and removed.

Alternatively, the conduit 102, 202, 302, 402 could be sewn in place during a surgical procedure or as an adjunct to another surgical procedure, such as coronary bypass grafting. Such a conduit would have a sewing ring instead of retention struts. The sewing ring could be made of Teflon™/polypropylene cloth, or some other similar material that is biocompatible and of sufficient strength to retain the conduit. Similarly, a balloon 424 connected to a stylet 418 could be used to control the shunt flow in the early period after device placement.

One method to manufacture an embodiment with the wire mesh design is to braid a biocompatible wire over a mandrel and/or over the conduit. A preferable wire is nitinol. If braided over the conduit, the wire braid could be welded to the conduit. The braid could also be used to sandwich a graft material between the conduit and the braid. The ends of the tubular braided structure could then be bunched together and inserted into the hollow interior of the deployment structure such as the threaded member, or inserted into a cap. Here, the braided ends would be potted or welded in place. The braided structure could then be heat treated to conform to the desired shape such as the discs that flatten out along the atrial septum.

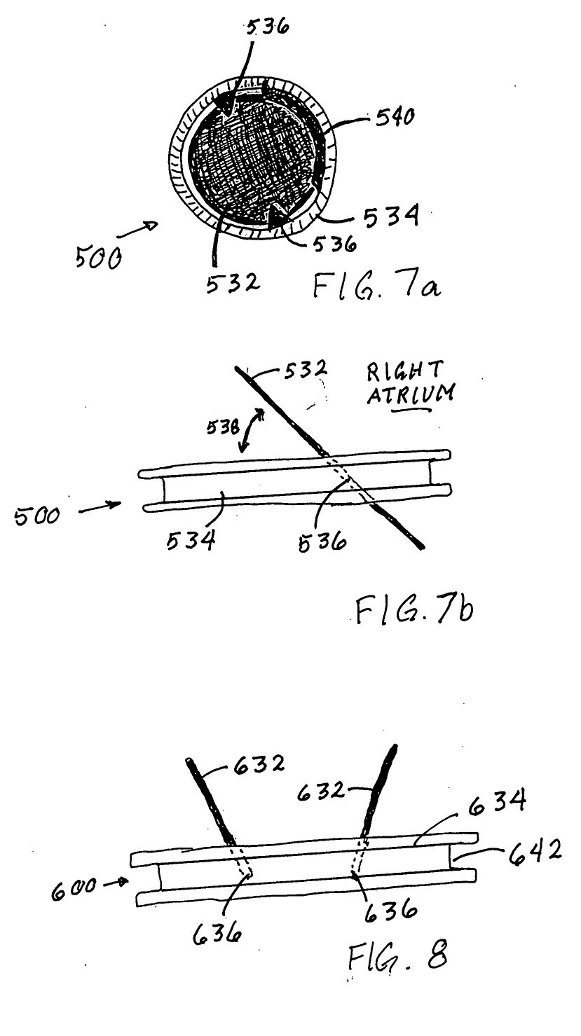

In another embodiment, a valve 500, shown in FIGS. 7a and 7b, is positioned within the conduit, rather than filters on each end. Preferably, the valve 500 has a preselected opening pressure, and therefore, shunting only occurs when the pressure gradient between the left and right atrium exceeds the valve opening pressure. The preferred method for creating this select opening pressure is through magnetic coupling of the valve occluder disc 532 and the valve housing 534.

The valve design shown in FIGS. 7a and 7b is that of a tilting disc. The disc 532 serves as an occluder to the flow path through the conduit. The disc 532 is contained and supported by a tubular housing 534 that spans the atrial septum. Pivots 536 or guides integrated into the housing 534 retain the disc 532 within the housing 534 and allow the disc 532 to pivot to the open position. In the open position, the disc 532 tilts open toward the right atrium, forming an angle 538 with the housing of 50 to 90 degrees. In the closed position, the disc 532 lies flat in the plane of the housing 534. A lip 540 protrudes inwardly from one side of the housing 534, keeping the disc 532 from inverting in the opposite direction. The valve 500 prevents emboli from right sided circulation from crossing over to the left sided circulation and causing a stroke (cryptogenic stroke).

To produce a selective opening pressure in this embodiment, the disc 532 is composed of a carbon coated permanently magnetized metal. Alternatively, the disc 532 could be made entirely of pyrolitic carbon with an integrated permanent magnet. The coating enhances durability and blood compatibility. Typical coatings include pyrolitic carbon or diamond-like coatings. The disc 532 magnetically couples to the magnetized protruding retention lip 540 of the housing 534. The force of this coupling determines the opening pressure of the valve 500. The opening pressure could be adjusted to an individual patient's need by changing the force of the magnetic coupling. The coupling force could allow a range of opening pressures at gradients from the left to the right atrium from 1 to 30 mm Hg, but open at a pressure gradient of at least 5 mm Hg. In some situations, it may not be desirable to have any magnetic coupling force, such that the valve opens whenever any pressure gradient between the right and left side exists. Alternatively, the disc 532 could be made of a plastic such as Isoplast™ or Delrin™, with an embedded permanent magnet. A plastic disc may not require the biocompatibility coating.

An alternative valve 600, as shown in FIG. 8, would be designed as a bileaflet structure. In this design, the magnetic coupling would occur between the two leaflets 632. The leaflets 632 would be of similar metallic coated construction as discussed above in the tilting disc design. The housing 634 and pivots/guides 636 are also made of a carbon coated metal, or entirely of carbon. The perimeter 642 of the housing 634 is slightly recessed to allow seating of the interatrial septum.

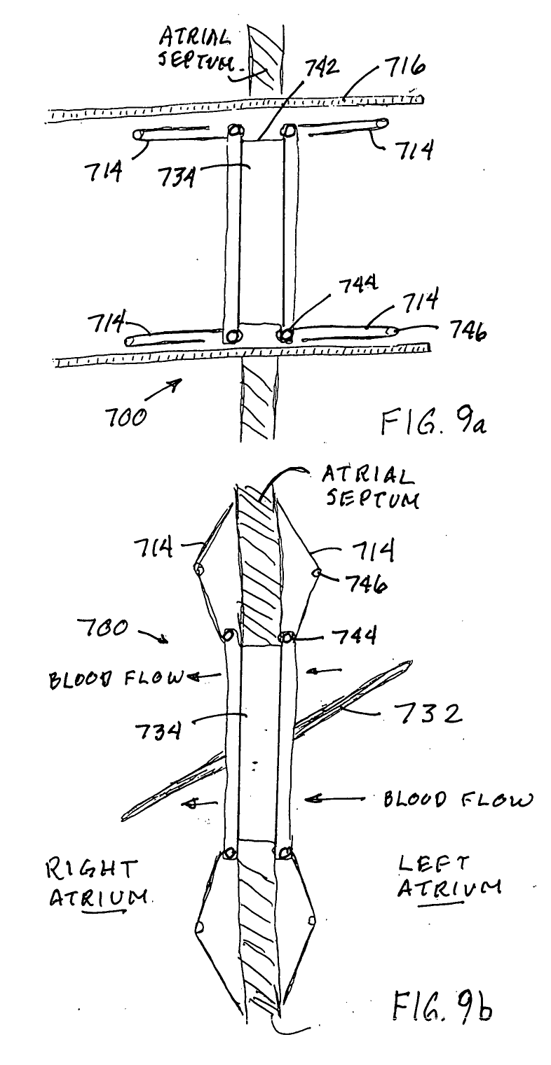

As shown in the embodiment 700 of FIGS. 9a and 9b, attached to at least two sides of the perimeter 742 of the housing 734 are retention struts 714 or arms for securing the housing 734 to each side of the atrial septum. The retention struts 714 are collapsible/deployable to facilitate delivery through a delivery catheter 716. When deployed, the struts 714 exert a slight force toward the septum on both sides, so as to pinch or clamp the valve 700 to the septum. The struts 714 may be metallic arms, with each arm 714 having a first spring joint 744 at the attachment of the arm 714 to the housing 734, and a second spring joint 746 midway down the arm 714. There would be several arms 714 on the housing 734. The arms 714 would fold back on themselves when contained within the delivery catheter 716 as shown in FIG. 9a, and unfold on each side of the septum when the delivery catheter 716 is withdrawn, to clamp the valve 700 in place as shown in FIG. 9b. A Dacron™ or Goretex™ mesh may span the retention struts 714, to allow cell growth and long term fixation to the septum.

Alternatively, a flap valve could be constructed from glutaraldehyde fixed bovine or porcine pericardium tissue. Such a valve would reduce anticoagulation needs. The pericardium tissue could be wrapped around a tubular structure, similar to the sheath 210 wrapped around the wire frame 212 in FIG. 4, with a flap occluding one end, similar to the occlusion disc 532 of the valve in FIG. 7a. A magnet could be sewn into the pericardium tissue to couple with a magnetized portion of the tubular structure 212.

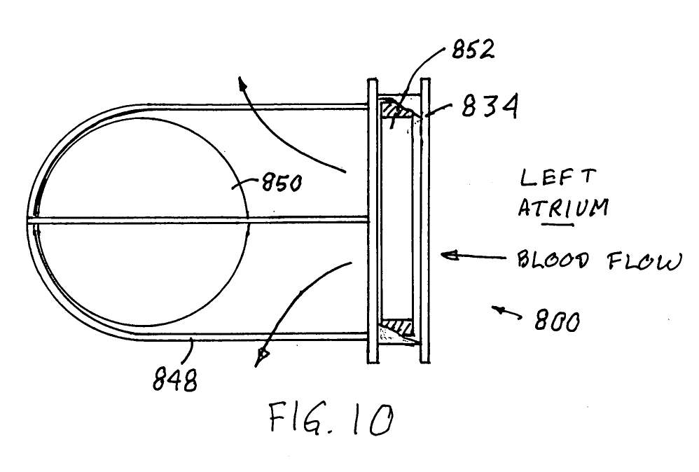

Another embodiment, shown in FIG. 10, is that of a caged ball device 800. The ball 850 is contained within a caged structure 848 on one end of a housing 834 that anchors to the septum. The cage 848 prevents the ball 850 from dislodging into the circulation, and it would be oriented into the right atrium. The ball 850 would be magnetized and coupled to a magnetic ring 852 in the housing 834. The magnetic ring 852, or another portion of the housing 834, would be slightly smaller than the ball 850, to prevent it from entering the left atrium.

While the particular invention as herein shown and disclosed in detail is fully capable of obtaining the objects and providing the advantages hereinbefore stated, it is to be understood that this disclosure is merely illustrative of the presently preferred embodiments of the invention and that no limitations are intended other than as described in the appended claims.

1. A device for treating heart failure, comprising a tubular conduit placed between the left atrium and the right atrium, said conduit being adapted to allow blood flow substantially from the left atrium to the right atrium.

2. The device recited in claim 1, wherein said conduit is adapted to allow blood flow only when pressure in the left atrium exceeds pressure in the right atrium.

3. The device recited in claim 1, further comprising an emboli barrier in said conduit.

4. The device recited in claim 1, further comprising a mechanism adapted to gradually open flow through said conduit.

5. The device recited in claim 1, wherein said conduit has a flow area large enough to substantially allow blood flow at normally experienced left atrial to right atrial differential pressures, but too small to substantially allow blood flow at normally experienced right atrial to left atrial differential pressures, to thereby substantially allow blood flow only from the left atrium to the right atrium.

6. The device recited in claim 5, wherein said conduit has a flow area not to exceed 2.0 cm².

7. The device recited in claim 1, further comprising an emboli barrier on at least one end of said conduit.

8. The device recited in claim 7, wherein said barrier comprises a wire mesh.

9. The device recited in claim 7, wherein said barrier comprises a coiled wire.

10. The device recited in claim 7, wherein said barrier comprises a porous structure.

11. The device recited in claim 7, further comprising a selectively inflatable and deflatable balloon in said conduit.

12. The device recited in claim 1, further comprising an occlusion member in said conduit, wherein:
said occlusion member is magnetically coupled to said conduit;
said magnetic coupling is designed to allow opening of said occlusion member at a selected pressure difference between the left atrium and the right atrium.

13. The device recited in claim 1, further comprising selectively deployable retention struts on said conduit.

14. A method for treating heart failure, comprising:
creating a hole in the interatrial septum of the heart;
placing a tubular conduit in said hole; and
allowing blood flow substantially from the left atrium to the right atrium.

15. The method recited in claim 14, further comprising gradually allowing an increase in said blood flow through said conduit from the left atrium to the right atrium.

16. The method recited in claim 15, further comprising:
providing at least one emboli barrier across said conduit;
providing a selectively inflatable and deflatable balloon;
placing said balloon within said conduit;
inflating said balloon when said conduit is within said hole, thereby occluding said conduit; and
gradually deflating said balloon within said conduit, thereby gradually allowing an increase in blood flow through said conduit from the left atrium to the right atrium.

17. The method recited in claim 14, further comprising:
providing a valve in said conduit; and
allowing blood to flow through said valve only when pressure in the left atrium exceeds pressure in the right atrium.
